(19) United States
(12) Patent Application Publication
(10) Pub. No.: US 2015/0154881 A1
(43) Pub. Date: Jun. 4, 2015

(54) Method for a Wellbeing Therapy Program

(71) Applicant: Jay P. Hotrum, VICTORVILLE, CA (US)

(72) Inventor: Jay P. Hotrum, VICTORVILLE, CA (US)

(21) Appl. No.: 14/094,777

(22) Filed: Dec. 2, 2013

(51) Int. Cl. G09B 19/00; A61K 36/38; A61K 31/625; A61K 36/16; A61K 31/616; A61K 31/167; A61K 36/258; A61K 31/192; A61K 31/137

(52) U.S. Cl. CPC G09B 19/00; A61K 31/192; A61K 36/38; A61K 31/625; A61K 31/137; A61K 31/616; A61K 31/167; A61K 36/258; A61K 36/16

(57) ABSTRACT

As one implementation example, a method for comforting and helping an addicted user is described which comprises referencing a first portion of an informational composition, the first portion comprising a first stage in a wellbeing sequence. The method also comprises establishing an addiction discontinuation point. The method also comprises on the next day after the discontinuation point, performing dishabituation steps comprised of drinking an amount of coffee upon waking, eating a breakfast and taking a vitamin, arriving home soon after performing a normal daily activity, and determining whether a sleeping aid will be necessary, and if deemed necessary, taking the sleeping aid after dinner. The method also comprises upon waking on the second day after the discontinuation point, referencing a second portion of the informational composition, which comprises a second stage in the wellbeing sequence.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to drug addiction treatment. More particularly, the invention relates to a multi-stage, self-detox treatment method.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that drug rehabilitation is a processes of medical or psychotherapeutic treatment, for dependency on psychoactive substances such as alcohol, prescription drugs, and street drugs such as cocaine, heroin or amphetamines. The general intent is to enable the patient to cease substance abuse, in order to avoid the psychological, legal, financial, social, and physical consequences that can be caused, especially by extreme abuse.

Typically, various types of programs offer help in drug rehabilitation, including: residential treatment (in-patient), out-patient, local support groups, extended care centers, recovery or sober houses, addiction counseling, mental health, orthomolecular medicine and medical care. Some rehab centers offer age- and gender-specific programs.

In many instances, methamphetamine also known as meth, ice, clouds crystal, glass, tik, N-methylamphetamine, methylamphetamine, and desoxyephedrine, is a psychostimulant of the phenethylamine and amphetamine class of psychoactive drugs. Methamphetamine is highly addictive. While the withdrawal itself may not be dangerous, withdrawal symptoms are common with heavy use and relapse is common.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a flowchart diagram of an exemplary drug treatment method, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present application or of any further application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

It is noted that according to USA law, all claims must be set forth as a coherent, cooperating set of limitations that work in functional combination to achieve a useful result as a whole. Accordingly, for any claim having functional limitations interpreted under 35 USC §112 (6) where the embodiment in question is implemented as a client-server system with a remote server located outside of the USA, each such recited function is intended to mean the function of combining, in a logical manner, the information of that claim limitation with at least one other limitation of the claim. For example, in client-server systems where certain information claimed under 35 USC §112 (6) is/(are) dependent on one or more remote servers located outside the USA, it is intended that each such recited function under 35 USC §112 (6) is to be interpreted as the function of the local system receiving the remotely generated information required by a locally implemented claim limitation, wherein the structures and or steps which enable, and breath life into the expression of such functions claimed under 35 USC §112 (6) are the corresponding steps and/or means located within the jurisdiction of the USA that receive and deliver that information to the client (e.g., without limitation, client-side processing and transmission networks in the USA). When this application is prosecuted or patented under a jurisdiction other than the USA, then “USA” in the foregoing should be replaced with the pertinent country or countries or legal organization(s) having enforceable patent infringement jurisdiction over the present application, and “35 USC §112 (6)” should be replaced with the closest corresponding statute in the patent laws of such pertinent country or countries or legal organization(s).

The present invention will now be described in detail with reference to embodiments thereof as illustrated in the accompanying drawings.

There are various types of drug treatment methods that may be provided by preferred embodiments of the present invention. In one embodiment of the present invention, the treatment method may include a multi-stage drug treatment regimen over a duration that helps teach a user how to self-detox from the drug. The treatment method may include a literary composition that helps explain the components of drug addiction to the user. The literary composition may further teach the user simple mental exercises for recognizing, addressing, and reversing triggers in the mind that create a desire for the drug. At least one medicinal composition may be integrated with the lessons from the literary composition to enhance the effectiveness of the treatment method. In another embodiment, the treatment method may be used to treat other addictions such as, without limitation, alcoholism, marijuana addiction, eating disorders, cigarette addiction, heroin addiction and common social disorders.

In some embodiments, the treatment method may provide a self-help styled drug treatment regimen that may be utilized while the user is still using the drug. The treatment method may combine a literary composition, which explains the elements of the drug addiction and teaches mental exercises for overcoming the drug addiction, with a medicinal portion that includes common over the counter pharmaceuticals, vitamins, and herbs for use in conjunction with the literary portion. In some embodiments, the treatment method may progress in a plurality of stages. An initial stage may comprise reading a first portion of the literary composition at a self-pace, until a selected drug termination date has been reached. The first portion of the literary composition may teach mental exercises and recommended medicinal compositions. A second stage may include reading a second portion of the literary composition while detoxifying from the drug. The second portion may be read in a predetermined sequence, such as one chapter every day. In one embodiment, the user may wait until twenty-four hours after the drug termination date to begin reading the second portion. Thereafter, each day may require reading a chapter, with each chapter providing exercises, advice, and confidence building measures. A third stage may comprise reading a third portion of the literary composition. The third portion may include a few chapters, chiefly dealing with paying back society by forgiving and contributing. The third portion also teaches the user to contemplate spirituality in life as a means for recovery.

In one embodiment of the present invention, the treatment method may include an initial Step of providing a multi-stage regimen for treating a drug addiction. The treatment method may include a multi-stage drug treatment regimen over a duration that helps teach a user how to self-detox from the drug. Those skilled in the art, in light of the present teachings, will recognize that the present invention may include elements of cognitive-behavioral therapy, which seeks to help patients to recognize, avoid and cope with situations in which they are most likely to relapse. This therapy takes time and is accomplished in discrete steps, whereby the patient may better understand the reasoning for the therapy.

An additional Step may include providing a literary composition that at least partially treats the drug addiction. In one embodiment, the literary composition may include, without limitation, “Quit Meth Now!” by Jay P. Hotrum. The literary portion may further comprise artistic depictions for helping to illustrate lessons. The method may then include a Step of selecting a drug discontinuation point for ceasing to utilize the drug. The drug discontinuation point may comprise an arbitrary date selected by the user, whereby the user may continue using the drug up to the drug discontinuation point, even while utilizing the treatment method. A next Step may include reading a first portion of the literary composition. In one embodiment, the first portion may include chapters 1 through 11. The first portion may comprise of at least one mental exercise for recognizing, addressing, and reversing triggers in the mind that create a desire for the drug. In one embodiment, the at least one mental exercise may include a Ball of Bad Memories exercise. The Ball of Bad Memories exercise may create a new memory system to counter the mind's efforts to utilize memories and senses that convince the user to relapse. In this manner, the efforts of the mind to force a relapse are countered by a mind trick that convinces the mind that the efforts are useless from this point on. In a further embodiment, the Ball of Bad Memories may teach that if there were a switch in the mind that could be turned on when the cravings for the drug are attempting to cause a relapse, a recovering user could flip the switch to make the cravings go away. The user may be taught how to build such a switch in the mind and to personally design the switch so it shuts down the cravings with a personalized ball in the mind that contains the worst memories and thoughts the user can think of about why they hate their addiction. In one embodiment, the at least one mental exercise may include a Define Your Motivation exercise. The Define Your Motivation exercise may create a new memory system to counter the mind's efforts to utilize memories and senses that convince the user to relapse. In this manner, the user may be taught a mind exercise that helps the user to define their motivations as to why they are attempting to quit the undesired addiction by creating a list of positive reasoning for breaking such a habit. The user may use this mental list of positive motivations as a tool to counter the mind's attempts to use cravings to create relapse.

In some embodiments, the first portion may further teach administering at least one medicinal composition for at least partially treating the drug addiction. The at least one medicinal portion may include over the counter pharmaceuticals, vitamins, and herbs that work in conjunction with the at least one mental exercise to help treat the drug addiction. The over the counter pharmaceuticals, vitamins and herbs may include, without limitation, one or more of the following: caffeine pills, ginseng, ibuprofen, aspirin, acetaminophen, gingko biloba, St. John's wort, multivitamins, Excedrin PM™ or other night time sleep aid, Excedrin Migraine™ or other migraine pain reliever, anti-nausea/anti-diarrheal medications such as Pepto-Bismol™ and/or SAM-E. A next Step may include reaching the drug discontinuation point. This Step may also include abstaining from the drug for at least twenty-four hours after the drug discontinuation point, prior to commencing with the next step. In some embodiments, another Step may include reading a second portion of the literary composition in a predetermined sequence. The predetermined sequence may include daily readings of a single chapter. Each chapter may teach the user exercises, advice, and confidence building measures. In one embodiment, the second portion may include chapters 12 through 25. In some embodiments, a final Step may include reading a third portion of the literary composition. The third portion may include a few chapters, chiefly dealing with paying back society by forgiving and contributing. The third portion also teaches the user to contemplate spirituality in life as a means for recovery. The third portion may include chapters 26 through 28.

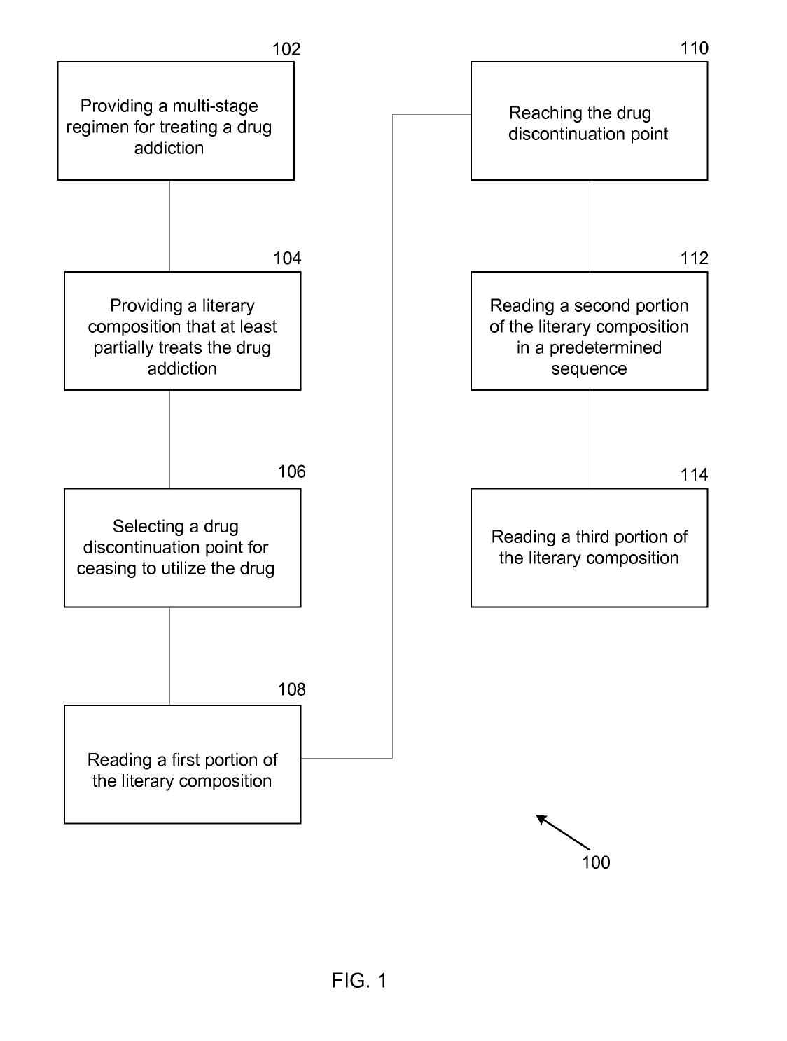

FIG. 1 illustrates a flowchart diagram of an exemplary treatment method, in accordance with an embodiment of the present invention. In the present embodiment, a treatment method 100 may provide a regimen for drug therapy. The drug may include methamphetamine and/or cocaine. However, in other embodiments, the drug may include, without limitation, tobacco, alcohol, marijuana, crack cocaine, heroin, and acid. In yet another embodiment, the addiction may include a social disorder, including, without limitation, overeating, gambling addiction, and depression. In some embodiments, the treatment method may utilize a combinative approach. The method may include both a literary composition, which explains the elements of the drug addiction and teaches mental exercises for overcoming the drug addiction, with a medicinal portion, which includes common over the counter pharmaceuticals, vitamins, and herbs for use in conjunction with the literary portion.

In one embodiment of the present invention, the treatment method may progress in a plurality of stages. Those skilled in the art, in light of the present teachings, will recognize that the present invention may include elements of cognitive-behavioral therapy, which seeks to help patients to recognize, avoid and cope with situations in which they are most likely to relapse. This therapy takes time and is accomplished in discrete steps, whereby the patient may better understand the reasoning for the therapy. An initial stage of the present invention may comprise reading a first portion of the literary composition at a self-pace, until a selected drug termination date has been reached. The first portion of the literary composition may teach mental exercises and recommended medicinal compositions. A second stage may include reading a second portion of the literary composition while detoxifying from the drug. The second portion may be read in a predetermined sequence, such as one chapter every day. In one embodiment, the user may wait until twenty-four hours after the drug termination date to begin reading the second portion. Thereafter, each day may require reading a chapter, with each chapter providing exercises, advice, and confidence building measures. A third stage may comprise reading a third portion of the literary composition. The third portion may include a few chapters, chiefly dealing with paying back society by forgiving and contributing. The third portion also teaches the user to contemplate spirituality in life as a means for recovery. Those skilled in the art, in light of the present teachings, will recognize that the treatment method may be most effective when used alone, or when combined with other types of drug treatments, including, without limitation, counseling, twelve-step programs, religious services, psychoanalytic treatment, and client-centered approaches.

In one embodiment of the present invention, the treatment method may include an initial Step 102 of providing a multi-stage regimen for treating a drug addiction. The treatment method may include a multi-stage drug treatment regimen over a duration that helps teach a user how to self-detox from the drug. In one embodiment of the present invention, the treatment method includes reading and understanding the entire content of the book Quit Meth Now! by Jay P. Hotrum, and following the suggestions and instructions in order of appearance. The addict must understand how they became addicted, how a chemical dependency changes one's own boidy chemistry, start a process that leads them to a quit-date, and then to start a 14-day self-detox that rids the body of the dependent drug while teaching the addict simple mind games that minimize cravings and attack the triggers in the mind that usually cause the addict to relapse.

An additional Step 104 may include providing a literary composition that at least partially treats the drug addiction. In one embodiment, the literary composition may include, without limitation, “Quit Meth Now!” by Jay P. Hotrum. The literary portion may further comprise artistic depictions for helping to illustrate lessons.

The method may then include a Step 106 of selecting a drug discontinuation point for ceasing to utilize the drug. The drug discontinuation point may comprise an arbitrary date selected by the user, whereby the user may continue using the drug up to the drug discontinuation point, even while utilizing the treatment method.

A next Step 108 may include reading a first portion of the literary composition. The first portion may comprise at least one mental exercise for recognizing, addressing, and reversing triggers in the mind that create a desire for the drug. In one embodiment, the at least one mental exercise may include a Ball of Bad Memories exercise. The Ball of Bad Memories exercise may create a new memory system to counter the mind's efforts to utilize memories and senses that convince the user to relapse. In this manner, the efforts of the mind to force a relapse are countered by a mind trick that convinces the mind that the efforts are useless from this point on. In a further embodiment, the Ball of Bad Memories may teach that if there were a switch in the mind that could be turned on when the cravings for the drug are attempting to cause a relapse, a recovering a user could flip the switch to make the cravings go away. The user may be taught how to build such a switch in the mind and to personally design the switch so it shuts down the cravings with a personalized ball in the mind that contains the worst memories and thoughts the user can think of about why they hate their addiction. In some embodiments, the first portion may further teach administering at least one medicinal composition for at least partially treating the drug addiction. The at least one medicinal portion may include over the counter pharmaceuticals, vitamins, and herbs that work in conjunction with the at least one mental exercise to help treat the drug addiction.

In one embodiment, the first portion may include chapters 1 through 11. Each chapter in the first portion is different, yet all follow a similar thread for helping the user break the drug addiction. For example, without limitation, Chapter 1 may introduce the author as an equal to the reader and explains how he became addicted and gains the trust of the reader who learns that the program was developed by someone who was addicted to the same drug they are addicted to. This offers hope and understanding that the program was designed by someone who walked in their shoes and knows what it feels like to be addicted to the drug that runs their own lives. In some embodiments, Chapter 2 may teach the user how the body and mind are simultaneously controlled by a chemical dependency and how the chemical dependency is what is keeping them addicted. Further, Chapter 2 may elucidate how the addiction is both physical and mental in nature. The user may learn about all phases of their addiction and why they became addicted in the first place. The user is taught about triggers in the mind that are keeping them addicted and how they work. The user gains trust for the writer by understanding that this is a one-on-one text that talks directly to the reader as if the user was being taught in a classroom setting. Finally, the user gains the understanding that the chapters are lessons that follow in order.

In one embodiment of the present invention, Chapter 3 may teach the user the entire treatment method so they know where they stand and what to expect as the program progresses. The user may learn that their exit from the drug world will require several different procedures attempted simultaneously. The user may learn that the treatment method is the expansion of an earlier project called QuitMeth and that the new program is a 2-phase, 3-step, 14-day program that guarantees success if they follow through with the instructions and suggestions and read through the entire text. In some embodiments, Chapter 4 may teach the user about the old QuitMeth program so that they understand the thinking behind the original program. This also builds up a new link-of-trust between the reader and the author. In some embodiments, Chapter 5 may teach the user how the treatment method is different from all other programs and that using God as a tool to kick an addiction is a bad idea. The treatment method may also teach why interventions fail so often. This chapter may then explain to the user how to pick a drug discontinuation date in advance and how to model the drug discontinuation date around their own expectations. The drug discontinuation date may be considered the most important element in the treatment method as it leads to the implementation of the 14-day self-detox. Chapter 5 may further teach the user the difference between a functioning addict and a non-functioning addict. Additional teachings include that the functioning addict should plan their exit by making sure that the worst days of withdrawal fall on days off of work, so as to minimize financial consequences. For example, without limitation, if the user runs a standard work week and has the weekend off, they would plan to quit on a Wednesday, so that the worst days fall around the time off of work. In some embodiments, Chapter 6 may teach the user how the human brain is made up of both a physical organ and the mind, and how both are addicted separately and collectively. Chapter 6 may further teach the user how to see the brain through the eyes of a child and how to recognize why and how the human brain has such a control over the addict. Additional lessons may include how the mind can store memories and use them as tools of manipulation against an addict that wants to quit drugs. Chapter 6 may additionally teach the user how mind control has kept them addicted and how it can also set them free.

In one embodiment of the present invention, Chapter 7 may teach the user how to create a “Ball of Bad Memories”. This technique comprises the biggest part of the program as it creates a new memory system to counter the mind's own efforts to convince the addict with his own memories and senses to relapse. The Ball of Bad Memories teaches that if there were a switch in the mind that a person could turn on when the cravings for drug are attempting to cause a relapse, a recovering addict would flip the switch to make the cravings go away. The user is taught how to build such a switch in the mind and to personally design the switch so it shuts down the cravings with a personalized ball in the mind that contains the worst memories and thoughts the user can think of about why they hate their addiction. The user is taught that the Ball of Bad Memories may comprise a counter attack to the minds own powers of persuasion. This is achieved by countering all cravings with a memory stampede of all of the horrible things that the drug has done to ruin their life. The user is taught to create a new file in their mind and to stick that file in a bubble, or ball, and to stack this ball on top of the highest box on the highest pallet in their mind. In that ball, the user is taught to remember anything and everything that makes them regret or hate their drug addiction and to focus on remembering each and every memory and to store them all in one place, in this ball of bad memories. In some embodiments, the user may be given a list of possible memories to include in the ball and told how to store it and to recall it when the mind uses any trigger to try to get the user to relapse. The ball can be recalled in an instance once the user has learned to use this technique. The user may further be taught that the attempts by the mind to cause a craving can be reversed when the user pulls up this memory set to counter the cravings. This works well from the start but as the user gets better at pulling up this memory bubble, the ball becomes more effective each time and quicker to stop the cravings. Eventually the mind may learn that the ball will be pulled and loses its power to persuade by inducing such cravings. This is a mind control technique for kicking any drug addiction being, utilizing the mind techniques to counter the triggers it creates. In a short period of time, the mind will soften its attempts to try to get the addict to relapse and eventually will stop this form of temptation altogether and focus on creating new ideas that work around the ball of bad memories. Finally, the user may be taught what to expect in the future so that the new types of triggers also have no effect on their decision to quit using forever.

In one embodiment of the present invention, Chapter 8 may help the user create a shopping list of items that will need to be purchased before the 14-day detox period begins. This way the user may collect the items as they prepare to reach the quit-date they have been thinking about. In some embodiments, the self-detox may not begin until the drug discontinuation date has arrived and these items on the list are purchased to relieve the withdrawal symptoms as the user recovers. In some embodiments, Chapter 9 may teach the user that the world surrounding them has to change because it is directly affecting the recovery process. The user is taught that all sights and sounds are part of the daily ritual and that changing the art may help to change the mind's perception that everything is normal. In some embodiments, changing the music that one listens to as well as the TV shows, movies and all art that one sees after opening their eyes in the morning, may provide a form of motivation to help convince the user's mind that all things that used to be normal are now changing. In some embodiments, Chapter 8 may suggest a list of movies and TV shows that the user could obtain and watch during the recovery process. These films and TV shows are hand-picked to sway the mind of the user towards believing a new life with meaning and substance is indeed within reach. Those skilled in the art will recognize that the shows, as well as the art and music, are a huge part of the subliminal teaching process that stands outside the teachings in the text itself. The user may additionally learn that the actual scope and gravity of the treatment is much bigger than the words written in the text.

In one embodiment of the present invention, Chapter 10 may teach the user three simple mind games that keep the recovering addict on a learning curve consistent with trying to stay clean long after the 14-day detox period has ended. The three techniques may be referred to as “The Three Stooges” in the text because they are so basic in nature. The first technique may be called “I'm Robbie Long” which teaches the user that they are indeed where they belong at each phase of their personal drug recovery, and that they have done whatever it takes to get to each part of the program on their own. The second technique may be called “you're on your own, you know” teaches the user that kicking this drug addiction can only be done if the user agrees that they are doing this on their own with the help of nobody else. The third technique may be called “Fat is good” to teach the user that they will be gaining weight once the addiction is past but that using weight gain as a trigger has to stop and be combated with the belief that fat is good, so that they can ignore any impulse to the belief that they were using the drug to stay thin. In some embodiments, Chapter 11 may teach the user the history of methamphetamine and methamphetamine facts prior to the drug discontinuation date. Those skilled in the art will recognize that Chapter 11 may be a “need to know” basis of methamphetamine history, as it applies to the recovering addict. The same reasoning holds true on the methamphetamine facts section. It is not an attempt to factually relay all points of methamphetamine and methamphetamine use but rather it is written as a fact sheet that will inspire the user to take into consideration certain points that will assist them in their recovery.

In one embodiment of the present invention, a next Step 110 may include reaching the drug discontinuation point. This Step may also include abstaining from the drug for at least twenty-four hours after the drug discontinuation point, prior to commencing with the next step. Prior to the drug discontinuation point, the user may continue using the drug while reading and performing the lessons in Chapters 1 through 11.

In one embodiment, a Step 112 may include reading a second portion of the literary composition in a predetermined sequence. The predetermined sequence may include daily readings of a single chapter. Each chapter may teach the user exercises, advice, and confidence building measures. In one embodiment, the second portion may include Chapters 12 through 25. In one embodiment, the second portion may comprise a 14-day self-detox program with instructions and suggestions that change from day to day, depending on which day the user is on after the drug discontinuation date. The 14-day self-detox may include the use of several over-the-counter pharmaceuticals, vitamins, and herbs that rotate in and out of the program as the needs of the user change from day to day through the detox period.

In some embodiments, each Chapter may comprise a lesson for a sequential day after the drug discontinuation date. For example, without limitation, Chapter 12 may be written for the first day of the self-detox part of the program. In some embodiments, the user may be taught to follow the guidelines learned in the previous chapters. The user may be given a pep-talk of how they will feel during the day and what they should think and do to stay clean as their body begins the detox period. The user is also reminded to use the Ball of Bad Memories as a tool to make it through the day. For example, without limitation, first day recommendations may be as follows:

1. Wake up with a cup of coffee.
2. Eat a breakfast and take the daily multivitamin purchased for the program.
3. Get home as soon as possible.
4. Consider taking the Excedrin PM™ after dinner to induce sleep for needed rest.
5. Read the next chapter as soon as you wake up.

Chapter 13 may include day 2 of the self-detox part of the treatment method. The user may be given instructions on the differences they will feel being on the second day without their drug of choice as it begins to leave the blood stream. User is given the pep-talk needed to proceed with the program and also is given a lesson on caffeine and how it can mask some of the effects of withdrawal from stimulants. Day 2 introduces the use of the over-the-counter migraine pills that include a pain reliever as well as a small dose of caffeine. The user may be taught about the differences and benefits of other over-the-counter pain relievers, aspirin, ibuprofen and acetaminophen. Each medicine is used separately as the user's needs require for pain relief, headaches and the muscle pain commonly associated with drug withdrawal. The user may be instructed to read all of the manufacturers recommendations and warnings on each and every label and not to exceed the recommended doses. The user may be also taught about using caffeine pills when needed and taught to break the pills in half to experiment with how the smaller dose of caffeine will stimulate the body, mind and nerve endings to partially mimic the effects of the missing drug of choice so that the detox period will be less noticeable, less painful, and therefore easier to cope with. Day 2 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to keep 3 over-the-counter pain remedies within reach during the day and use as needed. User is reminded the ibuprofen is used for pain in the muscles and that aspirin and acetaminophen are used for headache pain and body pain in general.
5. User is told to monitor their caffeine intake and to be aware of how much caffeine is already in their body if they decide to start using the caffeine pills and still advised to take them in half-doses of the manufacturer's recommendations. User needs to figure out how this pill affects them personally.
6. User is advised to be cautious if they include energy drinks into their own self-detox plan and to read the label and know how much caffeine is in the drink. It is very important that the user is well aware of excess caffeine use.
7. User is advised to drink plenty of fluids and to eat well throughout the day. Fluids include water and Gatorade for good hydration and rejuvenation.
8. Get home as soon as possible.
9. Consider taking the Excedrin PM™ after diner to induce sleep for needed rest.
10. Read the next chapter as soon as you wake up.

Chapter 14 may include day 3 of the self-detox part of the program. The user may be given a pep-talk to stay strong and use the entire mind techniques learned in previous chapters. The user may be taught that day 3 will begin a series of very difficult days that will involve flu-like symptoms. The user may introduced to the fact that using a Pepto-Bismol™ type of product will help relieve the additional withdrawal symptoms that may include upset stomach, vomiting and common diarrhea. The user may be introduced to the St. John's wort pill as an herbal remedy that will help to lift the spirits to help avoid falling into mood swings that include depression. Again, the inclusion of these items into the daily ritual is to mask the symptoms of withdrawal from the drug of choice while at the same time relieving the cravings that usually cause the addict to relapse because the pressures of withdrawal are the main reason for addiction in the first place. Day 3 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is advised to consider a Pepto-Bismol™ type of product should they decide that the flu-like symptoms would subside from its use. This is not so much as a part of the program as it is an addition to it.
7. User is told to monitor their caffeine intake and to be aware of how much caffeine is already in their body if they decide to start using the caffeine pills and still advised to take them in half-doses of the manufacturer's recommendations. User needs to figure out how this pill affects them personally.
8. User is advised to be cautious if they include energy drinks into their own self-detox plan and to read the label and know how much caffeine is in the drink. It is very important that the user is well aware of excess caffeine use.
9. User is advised to drink plenty of fluids and to eat well throughout the day. Fluids include water and Gatorade for good hydration and rejuvenation.
10. Get home as soon as possible.
11. Consider taking the Excedrin PM™ after diner to induce sleep for needed rest.
12. Read the next chapter as soon as you wake up. (pages 135-141)

Chapter 15 may comprise day 4 of the self-detox part of the treatment method. The user may be advised that most of the drug of choice is now completely out of the bloodstream and that the withdrawal symptoms are now their body and mind trying to adapt to a new chemical makeup that requires the user to learn how to function in a normal state. Those skilled in the art will recognize that since the user has not functioned this way in so long, the body and mind are still trying to reject the exclusion of the drug of choice and that pain and mind tricks are triggers intended to force a relapse. The user may be aware this would happen from previous lessons but is reminded that there are still a few horrible days ahead and to stay strong, focused, and to use the techniques from previous chapters to fend off any attempts to force a relapse through cravings. The user may be reminded to use all of the techniques from previous chapters to get through the day strong and focused. Day 4 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is advised to consider a Pepto-Bismol™ type of product should they decide that the flu-like symptoms would subside from its use. This is not so much as a part of the program as it is an addition to it.
7. User is told to monitor their caffeine intake and to be aware of how much caffeine is already in their body if they decide to start using the caffeine pills and still advised to take them in half-doses of the manufacturer's recommendations. User needs to figure out how this pill affects them personally.
8. User is advised to be cautious if they include energy drinks into their own self-detox plan and to read the label and know how much caffeine is in the drink. It is very important that the user is well aware of excess caffeine use.
9. User is advised to drink plenty of fluids and to eat well throughout the day. Fluids include water and Gatorade for good hydration and rejuvenation.
10. Get home as soon as possible.
11. Consider taking the Excedrin PM™ after diner to induce sleep for needed rest.
12. Read the next chapter as soon as you wake up. (pages 143-149)

Chapter 16 may include day 5 of the self-detox part of the program. The user may be made aware that this may very well be the toughest day of the program. Withdrawal is harsh and all of the techniques and suggestions laid out in the program to this point are put into play to minimize the cravings for the drug removed. The user may be taught to focus on their motivation for quitting and to realize that getting through the fifth day puts them one step closer to living their dream of a drug free lifestyle. The user may be reminded that the pain and discomfort is at its peak and that the rewards for getting through the day will last for the rest of their lives. On day 5, two items may be added to the daily ritual that would have made the user sick to their stomach had they taken these herbs in the previous four days. The first is a daily regimen of Ginseng to be taken midday so as not to upset the stomach by mixing with the vitamins taken in the morning. Ginseng may be used to reduce the stress caused by kicking a major addiction, once the drug of choice has left the bloodstream. Ginseng can help the body and mind during the reorganization phase of recovery by calming the nerves and allowing the user to feel as if everything is going to be all right. Those skilled in the art will recognize that Ginseng may be used to improve concentration while combating the side effects of a chemical withdrawal. The second addition today may include a Ginkgo biloba pill. This works in conjunction with the Ginseng to increase all of the effects mentioned thus far along with the herbs ability to increase mental alertness while minimizing depression often associated with the detoxification of any chemical dependency. Also, Ginkgo biloba herbs have been proven to help with nerve cell function and blood flow to the brain and central nervous system. This combination of herbs may create a super herbal remedy that addresses the major withdrawal symptoms at this point of recovery. The calming feelings that come over the recovering addict at this point may ease the stress and pressure normally associated with chemical withdrawal at this point in the program. Day 5 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is instructed to take a Ginseng herbal pill at midday and to include this into their daily regimen.
7. User is instructed to take a Ginkgo biloba pill with the Ginseng pill so the herbs can work together, again at midday.
8. User is advised to consider a Pepto-Bismol™ type of product should they decide that the flu-like symptoms would subside from its use. This is not so much as a part of the program as it is an addition to it.
9. User is told to monitor their caffeine intake and to be aware of how much caffeine is already in their body if they decide to start using the caffeine pills and still advised to take them in half-doses of the manufacturer's recommendations. User needs to figure out how this pill affects them personally.
10. User is advised to be cautious if they include energy drinks into their own self-detox plan and to read the label and know how much caffeine is in the drink. It is very important that the user is well aware of excess caffeine use.
11. User is advised to drink plenty of fluids and to eat well throughout the day. Fluids include water and Gatorade for good hydration and rejuvenation.
12. Get home as soon as possible.
13. Consider taking the Excedrin PM™ after diner to induce sleep for needed rest.
14. Read the next chapter as soon as you wake up.

Chapter 17 may include day 6 of the self-detox part of the program. The user may be reminded that the drug is absolutely out of their bloodstream and that they will now feel a sense of accomplishment but the ups and downs of this day will offer a tempting reminder that the power of the mind is still working against their goals of a drug free life. Those skilled in the art will recognize that triggers now include a belief that the user has kicked the drug of choice and could do it again if they want to relapse. The user may be reminded that tricks like this are just triggers that need to be shut down by using the ball of bad memories and the other techniques taught in previous chapters. The user may be reminded that they are no longer chemically dependent on the drug of choice and that all withdrawal symptoms felt at this point are due to the body and mind trying to readjust to the normal human chemical makeup. The user may be reminded of the first time they tried their drug and that the reverse of these initial feelings are the journey they are now on. The user may be reminded that they can do anything they want after the 14-day detox period, except for their drug of choice because they are addicted to that drug. In some embodiments, the user may be told that making it through day 6 may bring them to a part in the program that is all downhill after today. The user may be told that the detox strategy is the same as the day prior. Day 6 recommendations may be as follows:

1. Wake up with a cup of coffee.
2. User to start the day with migraine pills for pain relief and caffeine to minimize morning withdrawal symptoms. User is instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is instructed to take a Ginseng herbal pill at midday and to include this into their daily regimen.
7. User is instructed to take a Ginkgo biloba pill with the Ginseng pill so the herbs can work together, again at midday.
8. User is advised to consider a Pepto-Bismol™ type of product should they decide that the flu-like symptoms would subside from its use. This is not so much as a part of the program as it is an addition to it.
9. User is told to monitor their caffeine intake and to be aware of how much caffeine is already in their body if they decide to start using the caffeine pills and still advised to take them in half-doses of the manufacturer's recommendations. User needs to figure out how this pill affects them personally.
10. User is advised to be cautious if they include energy drinks into their own self-detox plan and to read the label and know how much caffeine is in the drink. It is very important that the user is well aware of excess caffeine use.
11. User is advised to drink plenty of fluids and to eat well throughout the day. Fluids include water and Gatorade for good hydration and rejuvenation.
12. Get home as soon as possible.
13. Consider taking the Excedrin PM™ after diner to induce sleep for needed rest.
14. Read the next chapter as soon as you wake up.

Chapter 18 may include day 7 of the self-detox part of the program. The user may be reminded that the worst parts of detox are now behind them. The focus may be on staying the course that got them to where they are now. The user may be taught that any move towards the drug of choice would involve repeating the horrible days they just lived through. In some embodiments, the user may be taught to feel a sense of accomplishment without giving in to new triggers that will surface as tricks to cause phony cravings that aim towards relapse. The user may be taught to use techniques in previous chapters to counter the triggers and reverse the trend that tries to temp the recovering addict with such triggers. Day 7 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is instructed to take a Ginseng herbal pill at midday and to include this into their daily regimen.
7. User is instructed to take a Ginkgo biloba pill with the Ginseng pill so the herbs can work together, again at midday.
8. User is advised to consider a Pepto-Bismol™ type of product should they decide that the flu-like symptoms would subside from its use. This is not so much as a part of the program as it is an addition to it.
9. User is told to monitor their caffeine intake and to be aware of how much caffeine is already in their body if they decide to start using the caffeine pills and still advised to take them in half-doses of the manufacturer's recommendations. User needs to figure out how this pill affects them personally.
10. User is advised to be cautious if they include energy drinks into their own self-detox plan and to read the label and know how much caffeine is in the drink. It is very important that the user is well aware of excess caffeine use.
11. User is advised to drink plenty of fluids and to eat well throughout the day. Fluids include water and Gatorade for good hydration and rejuvenation.
12. Get home as soon as possible.
13. Consider taking the Excedrin PM's after diner to induce sleep for needed rest.
14. Read the next chapter as soon as you wake up.

Chapter 19 may include day 8 of the self-detox part of the program. The user may be reminded that they are exactly where they want to be now, recovering from their drug and headed to a life, free of drug dependence. The user may be reminded that today will be just another day towards completing the self-detox period and that nothing is new, that they do not fully understand by this point. The user may be again encouraged to practice to perfection, the systems and methods that got them to this point in the program and to focus on the prize; a drug free lifestyle. The user may be given a pep-talk towards these personal goals. Day 8 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is instructed to take a Ginseng herbal pill at midday and to include this into their daily regimen.
7. User is instructed to take a Ginkgo biloba pill with the Ginseng pill so the herbs can work together, again at midday.
8. User is advised to consider a Pepto-Bismol™ type of product should they decide that the flu-like symptoms would subside from its use. This is not so much as a part of the program as it is an addition to it.
9. User is told to monitor their caffeine intake and to be aware of how much caffeine is already in their body if they decide to start using the caffeine pills and still advised to take them in half-doses of the manufacturer's recommendations. User needs to figure out how this pill affects them personally.
10. User is advised to be cautious if they include energy drinks into their own self-detox plan and to read the label and know how much caffeine is in the drink. It is very important that the user is well aware of excess caffeine use.
11. User is advised to drink plenty of fluids and to eat well throughout the day. Fluids include water and Gatorade for good hydration and rejuvenation.
12. Get home as soon as possible.
13. Consider taking the Excedrin PM™ after diner to induce sleep for needed rest.
14. Read the next chapter as soon as you wake up.

Chapter 20 may include day 9 of the self-detox part of the program. The user may be reminded that they are exactly where they want to be now, recovering from their drug and headed to a life, free of drug dependence. The user may be reminded that today will be just another day towards completing the self-detox period and that nothing is new, that they do not fully understand by this point. The user may be encouraged to practice to perfection, the systems and methods that got them to this point in the program and to focus on the prize; a drug free lifestyle. In some embodiments, the user may be given a pep-talk towards these personal goals. User is reminded that watching the movies and TV shows recommended earlier are essential pieces to their own personal program at this time. It's time to reprogram the brain into thinking differently about the world outside. The user may be told they can now back off on any of the self-detox recommendations as they see fit, to develop a program that suits them personally. Day 9 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is instructed to take a Ginseng herbal pill at midday and to include this into their daily regimen.
7. User is instructed to take a Ginkgo biloba pill with the Ginseng pill so the herbs can work together, again at midday.
8. User is advised to consider a Pepto-Bismol™ type of product should they decide that the flu-like symptoms would subside from its use. This is not so much as a part of the program as it is an addition to it.
9. User is told to monitor their caffeine intake and to be aware of how much caffeine is already in their body if they decide to start using the caffeine pills and still advised to take them in half-doses of the manufacturer's recommendations. User needs to figure out how this pill affects them personally.
10. User is advised to be cautious if they include energy drinks into their own self-detox plan and to read the label and know how much caffeine is in the drink. Very important that the user is well aware of excess caffeine use.
11. User is advised to drink plenty of fluids and to eat well throughout the day. Fluids include water and Gatorade for good hydration and rejuvenation.
12. Get home as soon as possible.
13. Consider taking the Excedrin PM™ after diner to induce sleep for needed rest.
14. Read the next chapter as soon as you wake up.

Chapter 21 may include day 10 of the self-detox part of the program. The user may be reminded that they are exactly where they want to be now, recovering from their drug and headed to a life, free of drug dependence. The user may be reminded that today will be just another day towards completing the self-detox period and that nothing is new, that they do not fully understand by this point. The user may be encouraged to practice to perfection, the systems and methods that got them to this point in the program and to focus on the prize; a drug free lifestyle. The user may be given a pep-talk towards these personal goals. User is reminded that watching the movies and TV shows recommended earlier are essential pieces to their own personal program at this time. The user may be told they can now back off on any of the self-detox recommendations as they see fit, to develop a program that suits them personally. Day 10 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is instructed to take a Ginseng herbal pill at midday and to include this into their daily regimen.
7. User is instructed to take a Ginkgo biloba pill with the Ginseng pill so the herbs can work together, again at midday.
8. User is advised to consider a Pepto-Bismol™ type of product should they decide that the flu-like symptoms would subside from its use. This is not so much as a part of the program as it is an addition to it.
9. User is told to monitor their caffeine intake and to be aware of how much caffeine is already in their body if they decide to start using the caffeine pills and still advised to take them in half-doses of the manufacturer's recommendations. User needs to figure out how this pill affects them personally.
10. User is advised to be cautious if they include energy drinks into their own self-detox plan and to read the label and know how much caffeine is in the drink. It is very important that the user is well aware of excess caffeine use.
11. User is advised to drink plenty of fluids and to eat well throughout the day. Fluids include water and Gatorade for good hydration and rejuvenation.
12. Get home as soon as possible.
13. Consider taking the Excedrin PM™ after diner to induce sleep for needed rest.
14. Read the next chapter as soon as you wake up.

Chapter 22 may include day 11 of the self-detox part of the program. The user may be reminded that they are exactly where they want to be now, recovering from their drug and headed to a life, free of drug dependence. The user may be reminded that today will be just another day towards completing the self-detox period and that nothing is new, that they do not fully understand by this point. The user may be again encouraged to practice to perfection, the systems and methods that got them to this point in the program and to focus on the prize; a drug free lifestyle. The user may be given a pep-talk towards these personal goals. The user may be reminded that watching the movies and TV shows recommended earlier are essential pieces to their own personal program at this time. It's time to reprogram the brain into thinking differently about the world outside. The user may be told they can now back off on any of the self-detox recommendations as they see fit to develop a program that suits them personally. Day 11 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is instructed to take a Ginseng herbal pill at midday and to include this into their daily regimen.
7. User is instructed to take a Ginkgo biloba pill with the Ginseng pill so the herbs can work together, again at midday.
8. User is advised to consider a Pepto-Bismol™ type of product should they decide that the flu-like symptoms would subside from its use. This is not so much as a part of the program as it is an addition to it.
9. User is told to monitor their caffeine intake and to be aware of how much caffeine is already in their body if they decide to start using the caffeine pills and still advised to take them in half-doses of the manufacturer's recommendations. User needs to figure out how this pill affects them personally.
10. User is advised to be cautious if they include energy drinks into their own self-detox plan and to read the label and know how much caffeine is in the drink. It is very important that the user is well aware of excess caffeine use.
11. User is advised to drink plenty of fluids and to eat well throughout the day. Fluids include water and Gatorade for good hydration and rejuvenation.
12. Get home as soon as possible.
13. Consider taking the Excedrin PM™ after diner to induce sleep for needed rest.
14. Read the next chapter as soon as you wake up.

Chapter 23 may include day 12 of the self-detox part of the program. The user may be reminded that they are exactly where they want to be now, recovering from their drug and headed to a life, free of drug dependence. The user may also be reminded that today will be just another day towards completing the self-detox period and that nothing is new, that they do not fully understand by this point. The user may be encouraged to practice to perfection, the systems and methods that got them to this point in the program and to focus on the prize; a drug free lifestyle. The user may be given a pep-talk towards these personal goals. The user may be reminded that watching the movies and TV shows recommended earlier are essential pieces to their own personal program at this time. IN some embodiments, the user may be told they can now back off on any of the self-detox recommendations as they see fit, to develop a program that suits them personally. The user may be reminded that this is nearing the end of the detox part of the program and that they may seem fully recovered but should focus on getting past the 14^{th} day of detox. This is an important element of the program now as the 14 days was the goal. Day 12 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is instructed to take a Ginseng herbal pill at midday and to include this into their daily regimen.
7. User is instructed to take a Ginkgo biloba pill with the Ginseng pill so the herbs can work together, again at midday.
8. User is advised to consider a Pepto-Bismol™ type of product should they decide that the flu-like symptoms would subside from its use. This is not so much as a part of the program as it is an addition to it.
9. User is told to monitor their caffeine intake and to be aware of how much caffeine is already in their body if they decide to start using the caffeine pills and still advised to take them in half-doses of the manufacturer's recommendations. User needs to figure out how this pill affects them personally.
10. User is advised to be cautious if they include energy drinks into their own self-detox plan and to read the label and know how much caffeine is in the drink. It is very important that the user is well aware of excess caffeine use.
11. User is advised to drink plenty of fluids and to eat well throughout the day. Fluids include water and Gatorade for good hydration and rejuvenation.
12. Get home as soon as possible.
13. Consider taking the Excedrin PM™ after diner to induce sleep for needed rest.
14. Read the next chapter as soon as you wake up.

Chapter 24 may include day 13 of the self-detox part of the program. The user may be reminded that they are exactly where they want to be now, recovering from their drug and headed to a life, free of drug dependence. The user may be reminded that today will be just another day towards completing the self-detox period and that nothing is new, that they do not fully understand by this point. The user may be encouraged to practice to perfection, the systems and methods that got them to this point in the program and to focus on the prize; a drug free lifestyle. In some embodiments, the user may be given a pep-talk towards these personal goals. The user may be reminded that watching the movies and TV shows recommended earlier are essential pieces to their own personal program at this time. It's time to reprogram the brain into thinking differently about the world outside. The user may be told they can now back off on any of the self-detox recommendations as they see fit, to develop a program that suits them personally. The user may be reminded that this is nearing the end of the detox part of the program and that they may seem fully recovered but should focus on getting past the 14th day of detox. This is an important element of the program now as the 14 days was the goal. The user may be told to focus on the future. Day 13 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is instructed to take a Ginseng herbal pill at midday and to include this into their daily regimen.
7. User is instructed to take a Ginkgo biloba pill with the Ginseng pill so the herbs can work together, again at midday.
8. User is advised to consider a Pepto-Bismol™ type of product should they decide that the flu-like symptoms would subside from its use. This is not so much as a part of the program as it is an addition to it.
9. User is told to monitor their caffeine intake and to be aware of how much caffeine is already in their body if they decide to start using the caffeine pills and still advised to take them in half-doses of the manufacturer's recommendations. User needs to figure out how this pill affects them personally.
10. User is advised to be cautious if they include energy drinks into their own self-detox plan and to read the label and know how much caffeine is in the drink. It is very important that the user is well aware of excess caffeine use.
11. User is advised to drink plenty of fluids and to eat well throughout the day. Fluids include water and Gatorade for good hydration and rejuvenation.
12. Get home as soon as possible.
13. Consider taking the Excedrin PM™ after diner to induce sleep for needed rest.
14. Read the next chapter as soon as you wake up.

Chapter 25 may include the final day of detox, day 14. The user may be reminded that they are exactly where they want to be now, recovering from their drug and headed to a life, free of drug dependence. The user may be reminded that today will be just another day towards completing the self-detox period and that nothing is new, that they do not fully understand by this point. The user may be encouraged to practice to perfection, the systems and methods that got them to this point in the program and to focus on the prize; a drug free lifestyle. The user may be given a pep-talk towards these personal goals. User is reminded that watching the movies and TV shows recommended earlier are essential pieces to their own personal program at this time. It's time to reprogram the brain into thinking differently about the world outside. The user may be told they can now back off on any of the self-detox recommendations as they see fit, to develop a program that suits them personally. The user may be reminded that this is nearing the end of the detox part of the program and that they may seem fully recovered but should focus on getting past the 14th day of detox. This is an important element of the program now as the 14 days was the goal. The user may be now taught to think about the future and what they expect out of life now that they are clean from their drug of choice. The user may be taught a strong lesson that pits the end of the detox period with the need to stay clean for the rest of their life by continuing on with using all of the mind techniques taught in previous chapters and encouraged to read the entire book again, from time to time. The user may be warned to not take this accomplishment with a grain of self, being that the mind will continue its quest to trick the addict back into relapse with hidden emotions and senses, and will wait for opportunities where the recovering addict is least likely to resist the temptation of cravings that can cause a relapse. Day 14 recommendations may be as follows:

1. Wake up with a cup of coffee.
2. User to start the day with migraine pills for pain relief and caffeine to minimize morning withdrawal symptoms. User instructed to take one pill at a time to help gauge the effects.
3. Eat a breakfast and take the daily multivitamin purchased for the program.
4. User is instructed to include the St. John's Wort pill into their daily regimen.
5. User is instructed to keep 3 over-the-counter pain remedies within reach during the day and use as needed. User is reminded the ibuprofen is used for pain in the muscles and that aspirin and acetaminophen are used for headache pain and body pain in general.
6. User is instructed to take a Ginseng herbal pill at midday and to include this into their daily regimen.
7. User is instructed to take a Ginkgo biloba pill with the Ginseng pill so the herbs can work together, again at midday.
8. User is advised to consider a Pepto-Bismol™ type of product should they decide that the flu-like symptoms would subside from its use. This is not so much as a part of the program as it is an addition to it.
9. User is told to monitor their caffeine intake and to be aware of how much caffeine is already in their body if they decide to start using the caffeine pills and still advised to take them in half-doses of the manufacturer's recommendations. User needs to figure out how this pill affects them personally.
10. User is advised to be cautious if they include energy drinks into their own self-detox plan and to read the label and know how much caffeine is in the drink. It is very important that the user is well aware of excess caffeine use.
11. User is advised to drink plenty of fluids and to eat well throughout the day. Fluids include water and Gatorade for good hydration and rejuvenation.
12. Get home as soon as possible.
13. Consider taking the Excedrin PM™ after diner to induce sleep for needed rest.
14. Read the next chapter as soon as you wake up.

A final Step 114 may include reading a third portion of the literary composition. The third portion may include a few chapters, chiefly dealing with paying back society by forgiving and contributing. The third portion also teaches the user to contemplate spirituality in life as a means for recovery. The third portion may include chapters 26 through 28. Each of these last three chapters may provide final advice to the user.

For example, without limitation, Chapter 26 may teach the recovering user about success and failure and the differences between how to walk away from the program clean and free of the chemical dependency that caused the addiction in the first place. The user may be taught that they have just kicked the most addictive drug on the planet and that should make them realize that they can now do anything they want in the world, except the drug they are addicted to. The user may be taught that they are now very special people in that they are starting from the gutter up and that anything they accomplish in their life will be something that came from the power they have within them, the power that they called on to kick this addiction in the first place. The user may be taught follow up advice and lessons that advise them that full recovery can take some time, depending on each individual and a host of factors, and that 6 months to a full year can see improvement at each step along the way. In some embodiments, the user may be taught that things are looking up and that the future is indeed bright, in every way. The user may be encouraged to start the design of who they would like to be and start taking steps to reach these goals. The user may be taught several new techniques to help them succeed in the future with a set of rules that make understanding how to live a drug free lifestyle a reality.

In Chapter 27, the user may be introduced to a Pay Back phase of the treatment method. In the Pay Back phase, the user may be reminded that the program is not complete until they can learn to forgive themself and walk away from all aspects of the drug life they've lived for so long, never to return. Payback may include a means to forgive oneself by paying back the world, for what they have taken away by not being a contributing member of a society that included them nonetheless. Payback may be referred to as Restitution for the Soul, and allows the user to solidify a personal plan at their leisure that will allow them to forgive the guilt that the normally recovering addict feels inside after kicking their addiction. The user may be encouraged to take time and to think up their own plan that they feel will be enough to let themselves off the hook once the task is completed. A recipe for personal forgiveness may be the final step in completing the treatment method and there may be no rush or time limit on deciding or implementing such a plan. The user may be taught a lesson on duty and the need to do the right things with the rest of their life, so they can eventually walk away completely and never again have to refer to themselves as a recovering addict. This is deep in philosophical insight in that the power to achieve actual success in the program may provide a feeling inside that all is indeed well, from this point on. Payback, or Restitution for the Soul, achieves this goal.

In some embodiments, Chapter 28 may be the last chapter in the literary portion. Chapter 28 may be geared towards teaching the recovering addict that there may or may not be a God or Gods in this world and that now may be a good time to find out what they really believe. Those skilled in the art, in light of the present teachings, will recognize that a recovering addict may be overwhelmed by an intense feeling of good and positive thoughts and this chapter encourages the user to look at the numbers of people who believe there is a God or other deities and to compare that with how they currently feel. The user may be told to examine the facts and to determine on their own if this is something they would like to pursue. The user may be told how to go about picking a religion or church that they would like to investigate and perhaps they will feel the magic that is fate, faith and destiny.

In one alternative embodiment, the treatment method may be utilized to treat social disorders, including, without limitation, overeating, gambling, lack of confidence, and phobias. In yet another alternative embodiment, the treatment method may incorporate a live lecture by Jay P. Hotrum at predetermined stages of the recovery. In yet another alternative embodiment, the second portion may be read after the drug discontinuation point, and read or re-readby the user at any point during the treatment method.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112 (6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing a multi-stage, self-detox drug treatment method according to the present invention will be apparent to those skilled in the art. Various aspects of the invention have been described above by way of illustration, and the specific embodiments disclosed are not intended to limit the invention to the particular forms disclosed. The particular implementation of the embodiments may vary depending upon the particular context or application. By way of example, and not limitation, the multi-stage, self-detox drug treatment method described in the foregoing were principally directed to multi-stage, self-detox drug treatment method implementations; however, similar techniques may instead be applied to social disorders, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The Abstract is provided to comply with 37 C.F.R. Section 1.72 (b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A method for comforting and helping an addicted user based on information in an informational composition, said informational composition comprising a multi-stage, self-detoxification wellbeing therapy program, the method taught by said informational composition comprising the steps of:
referencing a first portion of said informational composition, which comprises a first stage in a wellbeing sequence;
establishing an addiction discontinuation point;
on the next day after said discontinuation point, performing dishabituation steps comprised of:
drinking an amount of coffee upon waking;
eating a breakfast and taking a vitamin;
arriving home soon after performing a normal daily activity; and
determining whether a sleeping aid will be necessary, and if deemed necessary, taking the sleeping aid after dinner; and
upon waking on the second day after said discontinuation point, referencing a second portion of said informational composition, which comprises a second stage in a wellbeing sequence.

2. The method of claim 1, further comprising referencing a descriptive portion of said informational composition, the descriptive portion describing a mind technique to counter a craving trigger in the addicted user's mind that causes the addicted user to relapse.

3. The method of claim 1, further comprising referencing a motivational portion of said informational composition, the motivational portion encouraging the addicted user to pay back society for the loss accumulated during the addicted user's addictive behavior.

4. The method of claim 1, further comprising performing an additional dishabituation step on the second day or a subsequent day after said discontinuation point comprised of using a pain remedy.

5. The method of claim 4, wherein the pain remedy is Ibuprofen, Aspirin, or Acetaminophen.

6. The method of claim 1, further comprising performing an additional dishabituation step on the second day or a subsequent day after said discontinuation point comprised of drinking more than the addicted user's normal intake of fluids.

7. The method of claim 1, further comprising performing an additional dishabituation step on a subsequent day after said discontinuation point comprised of using a St. John's Wort supplement.

8. The method of claim 1, further comprising performing an additional dishabituation step on a fifth day after said discontinuation point comprised of using a Ginseng herbal supplement and a Ginkgo biloba supplement.

9. The method of claim 1, further comprising performing an additional dishabituation step on a subsequent day after said discontinuation point comprised of using a product to at least partially relieve digestive system upset.

10. The method of claim 9, wherein the product is an over-the-counter anti-nausea/anti-diarrheal medicine such as Pepto Bismol™.

11. The method of claim 1, further comprising performing an additional dishabituation step on a second day or a subsequent day after said discontinuation point comprised of using an over the counter remedy.

12. The method of claim 11, wherein the over the counter remedy is a pharmaceutical remedy, a vitamin supplement, or an herbal supplement.

13. The method of claim 1, wherein the wellbeing sequence comprises 14 stages.

14. The method of claim 2, wherein the mind technique comprises the addicted user creating a switch in the addicted user's mind that is turned on to make a craving go away, the switch being created by imagining an object and imagining storing memories which make the addicted user hate or regret the addicted user's addiction into the object, and then flipping the switch, thereby allowing the addicted user to mentally recall the object and the memories when the addicted user experiences the craving trigger.

15. The method of claim 1, wherein said informational composition is supplemented with audio or video media.

16. The method of claim 1, wherein said informational composition is a literary composition, an audio composition, or a video composition.

17. The method of claim 1, wherein the sleeping aid is Excedrin PM™.

18. A method for comforting and helping an addicted user based on information in an informational composition, said informational composition comprising a multi-stage, self-detoxification wellbeing therapy program, the method taught by said informational composition comprising the steps of:
referencing a first portion of said informational composition, which comprises a first stage in a wellbeing sequence;
establishing an addiction discontinuation point;
on the next day after said discontinuation point, performing dishabituation steps comprised of:
drinking an amount of coffee upon waking;
eating a breakfast and taking a vitamin;
arriving home soon after performing a normal daily activity; and
determining whether a sleeping aid will be necessary, and if deemed necessary, taking the sleeping aid after dinner;
upon waking on the second day after said discontinuation point, referencing a second portion of said informational composition, which comprises a second stage in a wellbeing sequence;
on the second day after said discontinuation point, performing additional dishabituation steps comprised of:
using a pain remedy; and
drinking more than the addicted user's normal intake of fluids; and
on a subsequent day after said discontinuation point, performing yet additional dishabituation steps comprised of:
using a St. John's Wort supplement; and
using a product to at least partially relieve digestive system upset.

19. A method for comforting and helping an addicted user based on information in an informational composition, said informational composition comprising a multi-stage, self-detoxification wellbeing therapy program, the method taught by said informational composition comprising:
steps for referencing a first portion of said informational composition, which comprises a first stage in a wellbeing sequence;
steps for establishing an addiction discontinuation point;
on the next day after said discontinuation point, step for performing dishabituation steps comprised of:
steps for drinking an amount of coffee upon waking;
steps for eating a breakfast and taking a vitamin;
steps for arriving home soon after performing a normal daily activity; and
steps for determining whether a sleeping aid will be necessary, and if deemed necessary, taking the sleeping aid after dinner; and
upon waking on the second day after said discontinuation point, steps for referencing a second portion of said informational composition, which comprises a second stage in a wellbeing sequence.
